(19) United States
(12) Patent Application Publication
(10) Pub. No.: US 2025/0222420 A1
(43) Pub. Date: Jul. 10, 2025

(54) BIODEGRADABLE MICROCAPSULES BASED ON CRYSTALLINE MATERIALS AND SYNTHESIS PROCESS

(71) Applicant: MIKROCAPS D.O.O, Ljubljana (SI)

(72) Inventors: Dejan Stefanec, Dobrova (SI); Aljosa Vrhunec, Ljubljana (SI); Domen Kranjc, Kamnik (SI)

(21) Appl. No.: 18/847,534

(22) PCT Filed: Mar. 15, 2022
(86) PCT No.: PCT/SI2022/050011

§ 371 Date: Sep. 16, 2024

(51) Int. Cl. B01J 13/04

(52) U.S. Cl. CPC B01J 13/04

(57) ABSTRACT

The present invention relates to a water dispersion of biodegradable microcapsules, with a membrane formed of a biodegradable crystalline material only, i.e. free of polymer framework, and synthesis thereof. Biodegradable microcapsules consist of a core material comprising at least one water-immiscible and water-insoluble active component, and a membrane that encloses this core material, the membrane consisting of a highly crystalline material only, wherein the crystalline material is a lipophilic biodegradable organic compound which is solid at room temperature and has a melting point equal to or above 40° C. Biodegradable microcapsules are in the form of aqueous dispersions and are used primarily for encapsulation of pesticides for use in pest control applications.

The present invention relates to biodegradable microcapsules with a membrane wall formed from a biodegradable crystalline material only and to the process of their synthesis. Biodegradable microcapsules of this invention are in a form of encapsulated particles in a water dispersion used primarily for encapsulation of pesticides, but also fragrances, active pharmaceutical ingredients, and other materials that are subsequently used in pest control, cosmetics, fabric softeners, detergents, paints and similar products.

### PRIOR ART

Microencapsulation is an established process in which an active ingredient is coated with a membrane, also known as a wall. The primary purpose of microencapsulation is to protect active ingredients inside the core from outside factors and prolonged or controlled release of these active ingredients. As such, microencapsulation also reduces mammalian toxicity of toxic substances and reduces the amount of active ingredient required. The end product of microencapsulation are microcapsules, composed of a core material, containing at least one active ingredient, and a wall. Typically, microcapsules range between 10⁻⁶ and 10⁻⁴ m in size.

The processes and technologies for the synthesis of microcapsules vary from field to field. The type of technology selected depends on the desired wall material, the properties of the core material and the end application. Microencapsulation techniques can generally be divided into chemical and physical techniques, depending on how the wall material is formed. Only a brief overview of microencapsulation techniques relevant to the invention are presented in this document, namely the synthesis of microcapsules from emulsions.

Firstly, a stable emulsion of two immiscible fluids is formed using surface active agents (surfactants) by dispersing one phase in the other. When a water and oil phase is used, water in oil (W/O) or oil in water (O/W) emulsions can be prepared. In this invention we will focus on O/W emulsions as the invention relates to the encapsulation of organic active compounds.

It is important to note that microcapsules obtained using different techniques have different properties. Chemical encapsulation techniques such as interfacial and suspension polymerization allow for the formation of more resistant microcapsules as most polymers are inert. Microcapsules obtained in these ways also enable the preparation of capsules with lower porosity as they can be more crosslinked to any desired degree. Such capsules are preferably used with volatile compounds such as fragrances and essential oils. In the case of physical and physical-chemical encapsulation techniques, such as phase separation, the membranes are not (as) crosslinked and as resistant because it is usually preferred for the wall material to slowly degrade and release the core material. The biodegradable microcapsules described in this invention are primarily synthesized from emulsions.

A commonly used method to prepare microcapsules from emulsions is interfacial polymerization. By adding one type of monomer to each phase, the reaction between monomers takes place in the interfacial phase of emulsion droplets. After a stable emulsion of satisfactory droplet size is formed, the process of polymerization is initiated (temperature, pH, catalyst . . . ), leading to the formation of the final polymer and trapping each droplet in the membrane. Interfacial polymerization and in-situ polymerization are frequently used methods to encapsulate pesticides. Both techniques allow long-lasting effectiveness of pesticides with decreased mammalian toxicity and offers protection from UV-degradation (EP2403333A1, U.S. Pat. Nos. 5,160,529A, 4,956,129A). In the examples described, polyurea and melamine formaldehyde microcapsules are frequently used.

Another method of microcapsule synthesis from emulsions is suspension polymerization. Here, the water (continuous) phase does not contain monomers, but a water-soluble initiator, which initiates polymerization at the interfacial interface. An example of suspension polymerization is the synthesis of polyacrylate microcapsules.

Lastly, phase separation is also a method of microcapsule synthesis from emulsions. In the phase separation technique, the active ingredient is either dissolved in a solution containing the wall material or is suspended in a solution of the wall material. The wall material is then induced to separate out as a separate phase by coacervation (i.e., by adding a non-solvent, by pH changing, by lowering the temperature, by adding a second polymer, or by a combination of these methods). This causes phase separation, with the newly formed phase positioning itself at the phase interface of emulsion droplets, encasing them in a membrane. This membrane is usually further chemically crosslinked in order to obtain stable material (WO 2013/174921).

However, higher crosslinking degree of the polymers results in the better stability of microcapsules and also a very slow biodegradability rate. The problem of non-degradable microplastic is rapidly gaining attention with the increasing focus on sustainable development. Microplastics (including microcapsules) made with crosslinked polymers, present in pesticides, cosmetics and personal care products, are especially problematic as they get washed away into the environment. They slowly degrade for centuries or, in the worst-case scenario, accumulate in wildlife. Although biodegradable microcapsules from natural materials do exist, they are not suitable for certain applications. They generally do not encapsulate the core material well enough and result in poorer mechanical properties and biodegradability.

Phase separation is also often employed and depending on the materials used it can also produce microcapsules of the matrix type. Whilst many materials used with this method are by themselves biodegradable, microcapsules obtained in this way most often undergo a final step of chemical crosslinking to ensure proper physical properties of the microcapsules and that the core material is well entrapped. This final step renders the microcapsules no longer biodegradable.

Some phase separation synthesis procedures make use of the phase transitions of materials, namely the transition from a crystalline solid to a melt, and vice versa. Under regulated conditions a material can first be dissolved in the core material and then be made to crystallize at the curved liquid/liquid interface upon cooling. This procedure produces microcapsules with a liquid core and a crystalline shell, so called crystalsomes. Crystalsomes produced currently employ long chain polymers such as poly(L-lactic acid) (PLLA), polyethylene, block copolymers of PLLA and polyethylene glycol, etc. (Mark C. Staub, Christopher Y. Li, Polymer 195 (2020) 122407; Hao Qi Et.Al. NATURE COMMUNICATIONS, 7 (2016) 10599). Crystalosomes with polymer chains are named also polymersomes and shows advantages in high mechanical stability, low permeability and slow releasing of the liquid core. (Rideau E. et all, Chem Soc Rev, 47 (2018) 8572).

The present invention uses highly crystalline material as a wall material, such as waxes and/or paraffins, fatty acids or polyethylene glycols with a melting point highly dependent on temperature to ensure that the chosen wall material is in liquid form at elevated temperatures, i.e. temperatures above the melting temperature of the wall material, and thus miscible with the core material. The wall material crystallizes out of the mixture at lower temperatures, i.e. temperatures below the melting temperature of the wall material, and forming microcapsules from a biodegradable crystalline material with encapsulated core material, and wherein said microcapsules are free of polymer framework and thus free of microplastics.

The standard OECD 301 closed bottle biodegradability test in an enclosed respirometer measuring the uptake of oxygen has proved that microcapsules from biodegradable crystalline material based on the present invention have improved biodegradability, are free of polymer framework, thus free of microplastics, and wherein the microcapsules retain characteristics comparable to those of the polymer-containing microcapsules.

### DETAILED DESCRIPTION OF INVENTION

In one embodiment, the present invention relates to a water dispersion of microcapsules with encapsulated core material, wherein the wall of the microcapsule is made from a biodegradable crystalline material only and is free of a polymer framework, i.e. free of microplastics, and wherein the microcapsules retain characteristics comparable to the characteristics of the microcapsules with the wall material comprising the polymer framework. In another embodiment, the present invention relates to a synthesis of a water dispersion of biodegradable microcapsules with encapsulated core material, i.e. to a process of encapsulation of the core material. Water dispersions of microcapsules prepared in accordance with the present invention are especially suitable for use in pest control applications, but also in personal care products and pharmaceuticals. The present invention is not limited to the above applications only and is suitable for the encapsulation of any active compound that allows for its encapsulation with the methods described in the present invention. The microencapsulation procedure described in the present invention allows for the encapsulation of a broad spectrum of organic compounds (or solutions) into microcapsules made from a biodegradable crystalline material.

The present invention is described in more detail below and presented in figures as follows:

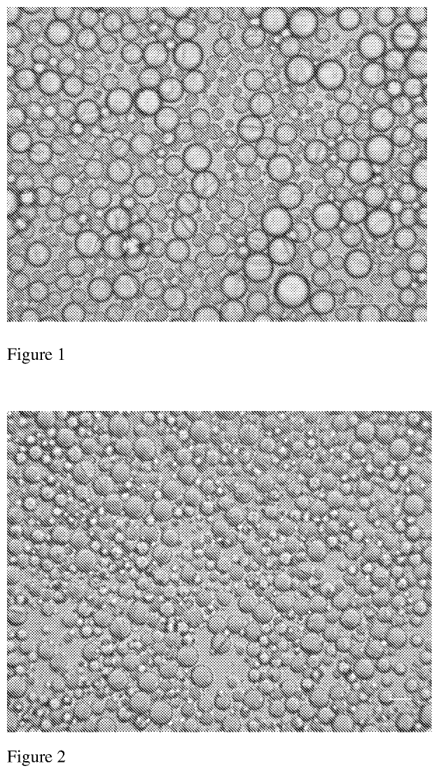

FIG. 1: Microcapsules based on present invention under a microscope (magnification 400×)

FIG. 2: Microscopy image of capsules after 28 days exposure at 60° C.

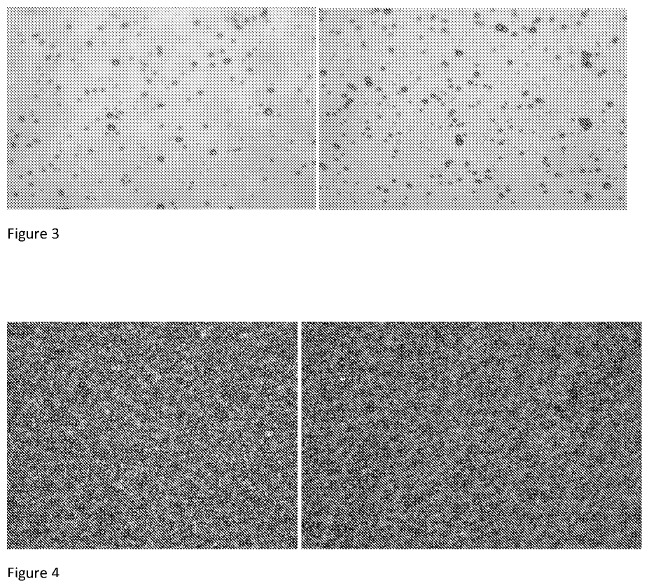

FIG. 3: picture of a glass slide with microcapsules based on present invention before (left picture) and after being rinsed thoroughly with tap water (right picture).

FIG. 4: Microscopy image of capsules with slow releasing: dried capsules right after water removing (left picture) and after one day at room temperature (right picture).

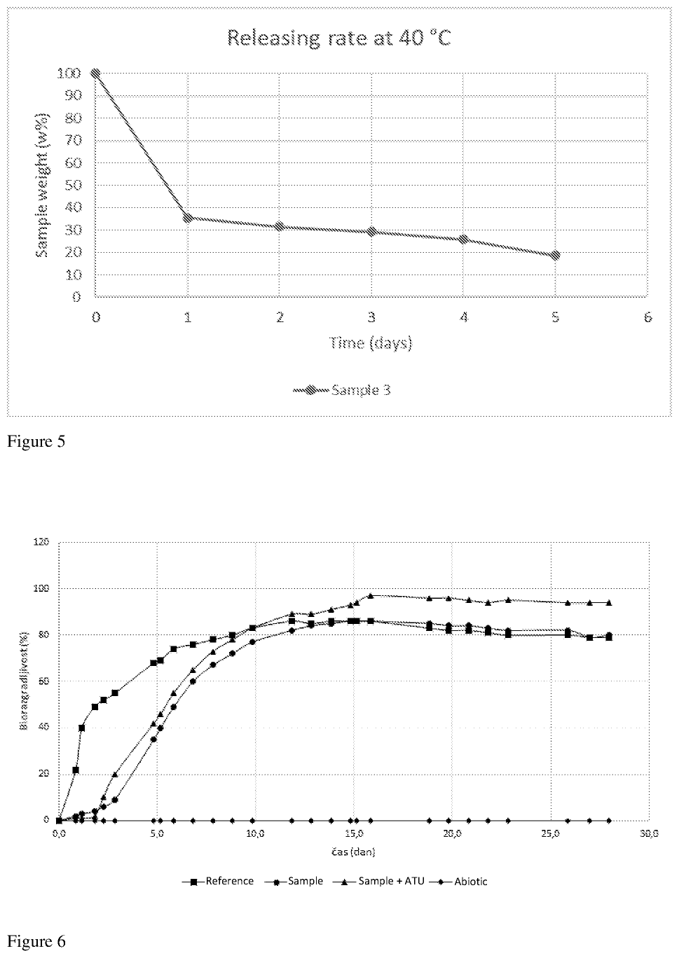

FIG. 5: Releasing rate by gravimetry.

FIG. 6 shows biodegradability results relative to time.

Water dispersion of biodegradable microcapsules wherein microcapsules consist of
a core material, comprising at least one active component in solid or liquid form and
a membrane as a wall material, encapsulating the core material, wherein the membrane is composed of a highly crystalline material only, wherein said crystalline material comprising at least one lipophilic biodegradable organic compound solid at room temperature and with a melting temperature equal to or above 40° C., wherein the diameter of the microcapsule is in the range of 1-50 μm.

The portion of the core material is 10-40% (w/w) of the end product that is a water dispersion (microcapsule slurry) and between 50% and 95% in a dry microcapsule.

Given that the synthesis of water dispersion of biodegradable microcapsules as described in the present invention is carried out by phase separation in an emulsion, the core material and thus the active component to be encapsulated preferably has the following properties:
it should be water-immiscible and water-insoluble, with the log P (partition coefficient) values of all components present in the core material above 2;
it should be inert to the wall material, meaning it should not chemically react with the wall material;
it should be in liquid state at elevated temperatures, i.e. above the melting temperature of the wall material
it should be miscible with the wall material at elevated temperatures, i.e. above the melting temperature of the wall material.

The core material may comprise a single active component or a mixture thereof.

The active component may be in solid form, wherein the melting temperature of the active component should be equal to or lower than the melting temperature of the wall material, thus enabling the miscibility of the core material with the melted wall material during the synthesis steps of water dispersion of the microcapsules.

The active component may be in liquid form or dissolved in an appropriate organic solvent, wherein the boiling temperature of the active component or the resulting solution should be above the highest temperature used in the synthesis steps of water dispersion of microcapsules, thus preventing the loss of the active component and/or the solvent during the synthesis steps.

Suitable active components are selected from pesticides, biocides, fragrances, pigments, pharmaceutical ingredients, phase change materials, etheric oils (e.g. eucalyptus oil, lavender oil, rose oil, common valerian oil, basil oil, juniper oil, citronella, lemon grass oil, and others), other oils (e.g. palm oil, coconut oil, castor oil, sunflower oil, olive oil, mineral oil) and photochromic materials. Preferably, the active components are selected from pesticides, including but not limited to insecticides selected from pyrethroids, carbamates or insecticides derived from plants.

Appropriate organic solvents should be water-immiscible, with log P values above 2. The organic solvent should be inert to the active component and to the wall material, meaning it should not chemically react with the active component and the wall material.

Suitable organic solvents are selected from hydrocarbon solvents (aliphatic and aromatic solvents, e.g. paraffin oils, waxes, mineral oils, petroleum distillates, white spirit), and vegetable oils (linseed oil, coconut oil, almond oil).

The selected highly crystalline material may be comprised of a single lipophilic biodegradable organic compound or a mixture thereof.

When selecting an appropriate wall material, its biodegradability and its melting temperature in correlation with the core material to be encapsulated, are important. The wall material should be solid at room temperature, thus its miscibility with the core material at room temperature is prevented.

When the core material is in solid form, the melting temperature of the wall material should be equal to or higher than the melting temperature of the solid core material, thus enabling miscibility of the melted core material with the melted wall material during the synthesis steps of water dispersion of the microcapsules.

When the core material is in liquid form, either per se or dissolved in the organic solvent, the melting temperature of the wall material should be lower than the boiling temperature of the liquid core material, for enabling the miscibility of the molten, i.e. liquid, wall material with the liquid core material.

Thus, the wall material is selected such that its miscibility with the liquid core material at temperatures above the melting temperature of the wall material, i.e. when the wall material is in liquid state, is enabled. Further, as the wall material is solid at room temperature and non-soluble in the core material (the core material is inert to the wall material), maximum crystallization of the wall material is enabled, when cooled in a controlled fashion to temperatures below 40° C., i.e. to temperatures below the melting temperature of the wall material.

Suitable wall material is chosen from highly crystalline waxes and/or paraffins, with melting temperature highly dependent on temperature. The preferred wall materials are highly crystalline waxes with melting temperatures in the range equal to or above 40° C. and equal to or below 80° C.

The biodegradable microcapsules free of the polymer framework are in a form of a water dispersion with a mass fraction of microcapsules between 5% and 40%.

With the correct selection of the wall material in correlation with the core material to be encapsulated, a stable water dispersion of biodegradable microcapsules free of microplastics is obtained, wherein microcapsules retain characteristics comparable to the characteristics of the microcapsules with the wall material comprising the polymer framework. This is particularly advantageous for use in pest control and biocide applications, where the tendency is to minimize the uptake of microplastics into the environment.

The synthesis of a water dispersion of biodegradable microcapsules from emulsions as described in the present invention comprises the following steps:
a) preparation of a liquid oil phase, wherein the core material to be encapsulated is pre-prepared separately to be in liquid state. When the core material is in liquid form at room temperature, it could be used per se or dissolved in the organic solvent. When the core material is in solid form at room temperature, it could be dissolved in organic solvent or melted. Pre-prepared core material in liquid state is mixed with the wall material at a temperature above the melting temperature of the wall material, whereby the wall material is melted and mixed with the liquid core material;
b) preparation of a water phase at a temperature above the melting temperature of the wall material, which includes a water solution of surface active ingredients;
c) preparation of a stable emulsion, wherein the oil phase is emulsified in the aqueous phase, and dispersed or emulsified droplets are formed of the size of the microcapsules being formed, and thus the stable formation of water dispersion of microcapsules;
d) controlled cooling of water dispersion to a temperature between 10° C. and 25° C., whereby the wall material crystallizes, forming a crystalline membrane and whereby a final water dispersion of biodegradable microcapsules is formed with a mass fraction between 5% and 40%.

Optionally, the synthesis of the water dispersion of biodegradable microcapsules may also include step e), where a stabilizer is added to the water dispersion of microcapsules to prevent the separation of microcapsules and water phase in the water dispersion, and/or pH regulators are added to set the pH value of the water dispersion to a desired value, mainly to better ensure the stability of the water dispersion or for easier use of the water dispersion in end products.

Step e) can follow step c), meaning the additions are added into the water dispersion prior to controlled cooling, or step e) can follow step d).

Preferably the temperature rage of the synthesis steps is in the range between T equal to or above 40° C. and equal to or below 90° C.

Choosing a wall material with a different melting temperature directly affects its crystallization during controlled cooling. When the water dispersion is cooled below the melting temperature of the wall material, it has a strong tendency to form crystals (it exhibits self-nucleating properties), leading to the eventual separation of the wall material from the carrier oil phase. This property of the wall material is utilized for the synthesis of biodegradable microcapsules. After preparing a suitable emulsion, the water dispersion is slowly cooled to temperatures between 10° C. and 25° C., causing the wall material to separate from the core material and crystalize at the phase interface. The result is a stable 5-40% water dispersion of microcapsules by mass of microcapsules.

Surface active agents prevent droplet coalescence when preparing an emulsion, resulting in a stable emulsion. Suitable surface-active agents are chosen amongst anionic, cationic and nonionic emulsifiers and stabilizers. Suitable anionic emulsifiers are sulphates, sulfonates, phosphates, and carboxylates, e.g. sodium lauryl sulphate, sodium dodecyl sulphate, sodium stearate and acrylates. Suitable cationic emulsifiers include, but are not limited to, quaternary ammonium salts. Suitable nonionic emulsifiers are all emulsifiers with an HLB value above 7.

Additionally, stabilizers dissolved in the water phase, acting as steric barriers preventing oil droplet coalescence, can be used. Suitable stabilizers include, but are not limited to, carboxymethylcellulose, polyvinyl alcohols, polyacrylic acids, polysorbates, polyethyleneimines, gum arabic, xanthan gum, glycerol monostearate and similar.

A dedicated homogenizer and/or a mechanic stirrer at high revolutions is used for the preparation of the emulsion.

The described procedure allows the synthesis of a range of biodegradable microcapsules with different properties, dependent on the membrane composition and the type and quantity of the wall material used. The type and quantity of the wall material used depends on the end use of the product. As already mentioned, the preferred use of water dispersions of microcapsules of the present invention is for use in pest control applications where water dispersion is sprayed onto the desired area and water is removed by natural evaporation. In the case of spontaneous releasing of the active material is required immediately after evaporation of water, only a low amount of the membrane material is used. Such capsules consist of 5 wt. % to 10 wt. % of the wall material in dried capsules. In the case of slow and homogenous releasing through time is needed after evaporation of water, higher amount of the membrane material is used (10 w % to 25 w % in the dried capsules).

### Examples

1. Synthesis of Biodegradable Microcapsules with Cypermethrin as the Core Material in a Water Dispersion of Polyvinyl Alcohol, Carboxymethylcellulose and Polyacrylic Acid with Paraffin Wax as the Wall Material

300 g water, 6 g polyvinyl alcohol (PVA) (Selvol E 205), 4 g carboxymethylcellulose (CMC) (Carbofix 5A) and 0.9 g polyacrylic acid (PAA) (Pemulen 1622) are mixed in a reactor, heated to 80° C. and mixed for 1 hour at 80° C. for PVA, CMC and PAA to completely dissolve. 1.75 g 10% NaOH is then added to neutralize PAA and the mixture is subsequently cooled to 58° C. Separately, 80 g cypermethrin is mixed with 40 g of an organic solvent (Exxsol D 60) and heated to 58° C. in a beaker. 30 g paraffin wax with a melting temperature of 58° C. is then added and mixed thoroughly. The obtained mixture is poured into the reactor and the mixing speed in the reactor is increased. Once the proper emulsion is obtained, the stirring of the mixture is ideally halted (to prevent further alterations of droplet size) and a cooling process is initiated. For cooling, the dispersion is either left to cool by itself to room temperature or cooled with the help of cold water to speed up the process. The mixture can be poured into final containers still hot or it can be cooled down beforehand. The end product produced in this way is a water dispersion of microcapsules with the following content: 67.6% (w/w) water solution of emulsifiers and stabilizers and 32.4% (w/w) microcapsules with the core material and the membrane. 17.3% (w/w) of end product is represented by cypermethrine, whereas cypermethrine represents 53.3% (w/w) of dry microcapsule weight. The ratio of the capsule membrane is 20% (w/w) of dry microcapsule weight.

2. Synthesis of Biodegradable Microcapsules with Cypermethrin as the Core Material in a Water Dispersion of Polyvinyl Alcohol and Xanthan Gum with Paraffin Wax as the Wall Material

286.5 g water, 12 g polyvinyl alcohol (PVA) (Selvol E 205) and 1.5 g xanthan gum (Kelzan AP) are mixed in a reactor, heated to 80° C. and mixed for 1 hour at 80° C. for PVA and xanthan gum to completely hydrate. The mixture is then cooled to 58° C. Separately, 80 g cypermethrin is mixed with 40 g of an organic solvent (Exxsol D 60) and heated to 58° C. in a beaker. 30 g paraffin wax with a melting temperature of 58° C. is then added and mixed thoroughly. The obtained mixture is poured into the reactor and the mixing speed in the reactor is increased. Once the proper emulsion is obtained, the stirring of the mixture is ideally halted (to prevent further alterations of droplet size) and a cooling process is initiated. For cooling, the dispersion is either left to cool by itself to room temperature or cooled with the help of cold water to speed up the process. The mixture can be poured into final containers still hot or it can be cooled down beforehand. The end product produced in this way is a water dispersion of microcapsules with the following content: 66.7% (w/w) water solution of emulsifiers and stabilizers and 33.3% (w/w) microcapsules with the core material and the membrane. 17.7% (w/w) of end product is represented by cypermethrine, whereas cypermethrine represents 53.3% (w/w) of dry microcapsule weight. The ratio of the capsule membrane is 20% (w/w) of dry microcapsule weight.

### 3. Synthesis of Biodegradable Microcapsules for Slow Releasing Properties Determination

In order to determine the releasing properties of capsules only volatile solvent was used as the core material without other active material. Microcapsules were prepared by the same procedure as in Example 1 by using 120 g of organic solvent (Exxsol D 60) and without cypermethrin.

### 4. Synthesis of Wall Material Dispersion for Use in Biodegradability Tests:

241.25 g water, 7.5 g polyvinyl alcohol (PVA) (Selvol E 205) and 1.25 g xanthan gum (Kelzan AP) are mixed in a reactor, heated to 80° C. and mixed for 1 hour at 80° C. for PVA and xanthan gum to completely hydrate. The mixture is then cooled to 58° C. 30 g paraffin wax with a melting temperature of 58° C. is then added into the reactor and mixed thoroughly, allowing the wax to melt. This new mixture is then mixed until emulsion droplets of the desired size of 1-3 μm are obtained. Once the proper emulsion is obtained, the stirring of the mixture is ideally halted (to prevent further alterations of droplet size) and a cooling process is initiated. For cooling, the dispersion is either left to cool by itself to room temperature or cooled with the help of cold water to speed up the process. The end product produced in this way is a water dispersion of crystalline wall material with the following content: 89.3% (w/w) water solution of emulsifiers and stabilizers and 10.7% (w/w) of crystalline wall material.

### Analyses of Microcapsules

### Procedure to Determine Microcapsule Shelf Life at Elevated Temperatures (Above Melting Temperature of Crystalline Material):

The microcapsule dispersion obtained by Example 1 is enclosed into a plastic container that corresponds to the product's final packaging and placed into an oven preheated to at least 2° C. higher than the melting point of the crystalline wall material (e.g. when using a wall material with the melting temperature of 58° C. the temperature of the oven is 60° C.). The sample is then checked for changes under a microscope after 1 month and visual changes of phase separation of the dispersion. Using this procedure we have concluded that there is no phase separation of the dispersion nor any visible changes of the microcapsules under optical microscopy (FIG. 2).

Procedure to Determine Microcapsule Adhesion to Polished Glass Panels, i.e. Simulating Adhesion of Capsules to Green Parts of Plants, which is Usually Problematic:

The microcapsule dispersion is diluted with water to make a 0.2% (w/w) solution. The newly obtained mixture is sprayed onto a polished glass panel to form a very thin film. The glass panel is left to dry at ambient temperature (20-25° C.). Once dry, the glass panel is exposed to running tap water (ca. 15° C.) for 15 minutes. The stream of water is strong enough to form a continuous flow as opposed to falling apart into individual drops. The glass panel is left to dry at ambient temperature and then inspected under a microscope—FIG. 3. The number of microcapsules left after the procedure is then compared to a control sample before the exposure to the stream of water The results are presented on FIG. 3, wherein the left figure represents adhered microcapsules of the control sample and the right figure represents the adhered microcapsules after the exposure to the stream of water. Using this procedure we have concluded that the adhesion of microcapsules obtained in ways described in the present invention exhibit same adhesion properties and therefore adhesion of capsules on plant leafs is also expected.

Procedure to determine the releasing rate of the core material Sample from Example 3 was used in the procedure for determine the releasing rate. Sample was put on a glass and dried to obtained capsules only. The results are shown on FIG. 4. The optical microscopy images were taken immediately (left) and after one day (right). The potential opening of capsules would be visible after one day in the case of capsules fast releasing, however based on the results there was no visible changes.

Next, 5 g of sample dispersion was weighted into petri dish and put in a oven with 40° C. The weight of the sample was checked every day in order to determine the releasing rate of the core material. The water from the dispersion evaporated in one day and dried capsules were weighted for next days. The results are presented in Table 1 and on FIG. 5. It was concluded that sample from experiment 3 obtained slow and homogenous releasing over the time proper for slow releasing applications.

TABLE 1

| Days | Sample 3 weight (wt. %) |
|---|---|
| 0 | 100 |
| 1 | 35.5 |
| 2 | 31.5 |
| 3 | 29.2 |
| 4 | 25.9 |
| 5 | 18.6 |

### OECD 301 Closed Bottle Biodegradability Test

To confirm the biodegradability of the microcapsule sample obtained as described in example 1 in the present invention, a biodegradability test following OECD 301 F (Determination of ready biodegradability in a closed respirometer by measuring oxygen consumption) was carried out. The test was placed on the list of suitable methods for testing biodegradability of microplastics by the European Chemicals Agency (ECHA).

### a) Description of the Test Method

Microcapsule sample 1 was prepared by filtering the water dispersion of microcapsules and rinsing it with water to remove water-soluble components (emulsifiers, non-reacted reactants). The sample obtained in this way was then dried at 80° C. to remove the fragrance from the microcapsule core. The dry solid remains of the sample consisted only of the microcapsule membrane and served as the sample to be used in the biodegradability test.

For the test, we used activated sludge from a municipal wastewater treatment plant. The sludge was collected the day before the biodegradability test, washed at least 5 times with tap water, and its concentration (mg MLVSS/L) was determined by filtering 20 mL of a suspension of activated sludge with black ribbon filter paper. The sludge was then placed in a climate chamber (22±2° C.), where it was stirred and aerated until it was used.

The performed biodegradability assessment test is one of the optional tests for determination of ready biodegradability. It is based on the measurement of oxygen consumption in a closed respirometer, where biodegradation is measured indirectly through oxygen consumption at a constant temperature of 20±1° C. for 28 days. The concentration of activated sludge in the test was 30 mg/L. It was not necessary to adjust the pH before the test because the pH of the test mixture was 7.8±0.0. (The optimal range is between 6-8.) In parallel with that, we also performed a test with a reference substance (sodium acetate), which confirmed the activity of microorganisms and regular conditions for biodegradation throughout the test. Abiotic degradation was also determined in a system without the addition of activated sludge to the mixture, at the same time chemically sterilized by adding HgCl₂. The concentration of the sample in the test was 0.045 vol. % (COD=100 mg/L). Abiotic degradation was also measured with the same sample concentration. Each test was performed in parallel. The test with the same sample concentration and with added allylthiourea—ATU (4 mL/L) as a nitrification inhibitor was also performed. Thus, the measured oxygen consumption was proven to be due to the (bio) degradation of the sample and not to nitrification.

### b) Test Results

Average chemical oxygen demand (COD) was determined. pH of the microcapsule sample was 7.2±0.1.

| Sample repetition | COD (mg/L) |
|---|---|
| 1 | 217 756 |
| 2 | 203 616 |
| 3 | 225 432 |
| 4 | 230 684 |
| Average | 219 372 ± 11 770 |

In parallel, we measured oxygen consumption in the blank sample, the test with the reference substance (sodium acetate), the sample and in the abiotic sample. We also checked the oxygen consumption in the blank sample and the sample with added ATU to make sure that nitrification (oxidation of ammonium, which is not (bio)degradation of the sample) and consequent oxygen consumption does not occur. Initial pH of the mixture was in the range of 7.7±0.3 so according to the standard ISO procedure the pH regulation was not necessary, and pH of the sample had no effect on the biodegradation.

The results showed that the reference compound degraded well. After only 5 days, more than 60% was degraded. This confirmed the activity of microorganisms, the adequacy of the test and the validity of the results. Sample also degraded well in the test. In 28 days of the test is reached 86±0% degradation. This level of degradation was reached on the 14th day of the test, after a 3-day lag phase. Rapid degradation of the sample was also confirmed in the test with the sample with added ATU (added to prevent nitrification and consequent oxygen consumption), as in this case complete degradation of the sample (94±2%) was achieved. Since the degradation in the sample with ATU is comparable to the sample without the ATU added and even higher, it can be concluded that the difference between the two curves is due to the experimental error or the principle of the test, and nitrification did not occur. So as correct value the one without ATU can be taken (86%) with no abiotic degradation (0%). It can be concluded that the degradation is result of the microorganisms' activity (biodegradation). The results are presented in FIG. 6.

1. Water dispersion of biodegradable microcapsules with a diameter in the range of 1-50 μm, wherein the microcapsules comprises:
a core material, comprising at least one active component in solid or liquid form and
a membrane as a wall material, encapsulating the core material, wherein the membrane is composed of a highly crystalline material only, wherein said crystalline material comprising at least one lipophilic biodegradable organic compound solid at room temperature and with a melting temperature equal to or above 40° C., or a mixture thereof,
wherein the core material is water-immiscible and water-insoluble, with log P values of all components present in the core material above 2,
wherein the core material is inert to the wall material, meaning it should not chemically react with the wall material,
wherein the wall material is free of polymer framework and
wherein a mass fraction of biodegradable microcapsules in water dispersion is between 5% and 40%.

2. Water dispersion of biodegradable microcapsules according to claim 1, wherein the wall material represents 5 to 95%, preferably 5 to 25% by weight relative to the weight of the core material.

3. Water dispersion of biodegradable microcapsules according to claim 1, wherein the core material comprises a single active component or a mixture thereof.

4. Water dispersion of biodegradable microcapsules according to claim 3, wherein the active component is in solid form with the melting temperature equal to or lower than the melting temperature of the wall material.

5. Water dispersion of biodegradable microcapsules according to claim 3, wherein the active component is in liquid form per se or dissolved in an organic solvent.

6. Water dispersion of biodegradable microcapsules according to claim 5, wherein the organic solvent is water-immiscible, with log P values above 2, and wherein the organic solvent is inert to the active component and to the wall material.

7. Water dispersion of biodegradable microcapsules according to claim 1, wherein the active component is selected from pesticides, biocides, fragrances, pigments, pharmaceutical ingredients, phase change materials, etheric oils including, but not limited to, eucalyptus oil, lavender oil, rose oil, common valerian oil, basil oil, juniper oil, citronella, lemon grass oil and others, and other oils including, but not limited to palm oil, coconut oil, castor oil, sunflower oil, olive oil and mineral oil, and photochromic materials.

8. Water dispersion of biodegradable microcapsules according to claim 1, wherein the wall material is selected from highly crystalline waxes and/or paraffins, with a melting temperature in the range equal to or above 40° C. and equal to or below 80° C.

9. A synthesis process of water dispersion of biodegradable microcapsules according to claim 1 comprising the following steps:
a) preparation of a liquid oil phase, wherein the core material to be encapsulated is pre-prepared to be in liquid state, pre-prepared core material in liquid state is mixed with the wall material at a temperature in the range equal to or above 40° C. and equal to or below 90° C., wherein the wall material is melted and mixed with the liquid core material;
b) preparation of a water phase at a temperature above the melting temperature of the wall material, which includes a water solution of biodegradable surface active ingredients;
c) preparation of a stable emulsion, wherein the oil phase is emulsified in the aqueous phase, forming dispersed or emulsified droplets the size of the microcapsules being formed;
d) controlled cooling of the aqueous dispersion to a temperature between 10° C. and 25° C., whereby the wall material crystallizes, forming a crystalline membrane and whereby a final aqueous dispersion of biodegradable microcapsules is formed with a mass fraction between 5% and 40%.

10. The synthesis process according to claim 9, wherein the process optionally includes a step e), where a stabilizer is added to the water dispersion of microcapsules to prevent the separation of microcapsules and water phase in the water dispersion, and/or pH regulators are added to set the pH value of the water dispersion to a desired value, for ensuring the stability of the water dispersion or for easier use of the water dispersion in end products.

11. The synthesis process according to claim 9, wherein step e) follows step c), meaning the additions are added into the water dispersion prior to controlled cooling, or step e) follows step d).

12. The synthesis process according to claim 9, wherein the surface-active agents are selected from anionic, cationic, and nonionic emulsifiers and stabilizers, wherein the anionic emulsifiers are selected from sulphates, sulfonates, phosphates, and carboxylates, the cationic emulsifiers are quaternary ammonium salts, the nonionic emulsifiers are emulsifiers with an HLB value above 7, the stabilizers are selected from carboxymethylcellulose, polyvinyl alcohols, polyacrylic acid, polysorbates, polyethyleneimines, xanthan gum, gum Arabic and glycerol monostearate.

13. Water dispersion of biodegradable microcapsules according to claim 1 for use in pest control applications.
